(19) United States
(12) Patent Application Publication
(10) Pub. No.: US 2016/0324920 A1
(43) Pub. Date: Nov. 10, 2016

(54) Cellular Specific Therapeutic Prion

(71) Applicant: Ian Gill Bemis, Bowie, MD (US)

(72) Inventor: Ian Gill Bemis, Bowie, MD (US)

(21) Appl. No.: 15/192,030

(22) Filed: Jun. 24, 2016

(51) Int. Cl. A61K 38/16

(52) U.S. Cl. CPC A61K 38/164

(57) ABSTRACT

A method of inducing cell death or apoptosis in certain (undesirable) cells, such as cancerous cells or pathogenic microorganisms. The method to be described utilizes self-replicating prion-like misfolded isoforms of a cellular protein (which may be specific to a particular type of cell) to specifically target certain undesired cells for destruction.

### INVENTION BACKGROUND

1. Field of Invention

The invention to be described, specified, and documented in this United States Non-Provisional Utility Patent Application, is a new and useful means of utilizing certain naturally or synthetically misfolded, self-replicating (prion-like) isoforms of proteins, specific to a certain form or type of cellular organism to cause the death of that specific organism, or a population, mass, or tissue of consisting of such. While many embodiments of this invention may be applicable to a wide variety of (undesirable) cellular organisms, the misfolded isoforms of the natural cellular proteins produced specifically by these cells may take various forms with respect to the forms of cell or population of cells targeted for destruction by the individual (intentionally) misfolded protein(s).

2. Invention Background

The central nervous systems of mammals contain a form of native protein, Major Prion Protein, a Protease Resistant Protein, abbreviated variously as PrP, or PrP^{C}. The PrP protein may also occur, through natural or artificial means, as various misfolded isoforms, or PrP^{SC} which will cause other, common state PrP^{C} proteins to become misfolded by conversion to the isoform PrP^{SC}, the accumulation of which, causes a rapid and exponentially progressive pattern of cell death in several biological systems, primarily neural tissue. The macroscopic implications of the cell death caused by the rapid conversion of PrP^{C} to PrP^{SC} manifests in several mammals, as a separate class of pathogenic disease or Prion Disease or TSE (Transmissible Spongiform Encephalopathy), a group of invariable fatal neurodegenerative diseases, that typically occur sporadically in individuals, with no known pathogen exposure, or by genetic predisposition.

Clinical cases of TSE (prion disease) has been observed under medical, zoological and veterinary circumstances, afflicting a variety of domesticated mammals as well as mammalian wildlife, including but not limited to Cattle in the form of Bovine Spongiform Encephalopathy (BSE), Felines in the form of Feline Spongiform Encephalopathy, Mink in the form of Transmissible Mink Encephalopathy, Cervids (such as Deer, Moose and Elk) in the form of Chronic Wasting Disease (CWD), Goat and Sheep may contract Scrapie, which may be of acquired transmission or occur sporadically, as is the case with the NOR-98 variant of Scrapie, as well as humans in the form of Creutzfeldt-Jakob Disease (CJD), Variant Creutzfeldt-Jakob Disease (vCJD), Iatrogenic Creutzfeldt-Jakob Disease (iCJD), Familial Creutzfeldt-Jakob Disease (fCJD), Gerstmann-Sträussler-Scheinker Syndrome (GSS), Fatal Familial Insomnia (FFI), Sporadic Fatal Insomnia (SFI), and Kuru.

Self-replicating proteins of similar proteopathic function as to those found to cause disease in infected mammals have been known to exist in other lifeforms such as the fungal species including Podospora anserina and Saccharomyces cerevisiae, where the aggregation of these misfolded proteins may (under certain conditions) benefit the host.

Human cases of iatrogenic transmission of Creutzfeldt-Jakob Disease (CJD in any form being the most common form of TSE in humans) under medical settings through transplant of contaminated tissue and use of contaminated medical instruments. Under any medical, veterinary, or zoological circumstances, TSE or prion disease remains an incurable, untreatable, invariably lethal condition marked by a rapid decline in neurological function, leading to permanent disability, and ultimately rapid mortality. In a timeframe typically measured in weeks or months from onset of first symptoms, any form of TSE, procures a 100% consistent prognosis of assured mortality, under conspicuously painful and distressing conditions.

Various attempts to prevent, treat, or cure any form of TSE have yielded few practical results. A prior attempt to procure humoral immunity from the transmission of certain forms of PrP^{SC} by means of introduction or immunogenic exposure to a non-pathogenic variant of PrP, which is expressed or carried by attenuated strains of the bacterium Shigella and/or Salmonella. This work has resulted in the grant of U.S. Pat. No. 8,685,718, Titled Mucosal Immunization to Prevent Prion Infection, invented by Thomas Wisniewski of Staten Island, N.Y., Einar M. Sigurdsson of New York, N.Y., Jose Alejandro Chabalgoity of Montevideo Uruguay, Fernando R. Goni of Montevideo, Uruguay and Blas Frangione of New York, N.Y., the rights pertaining to such are assigned to New York University in New York, N.Y.

Research relating to the various forms of prion or prion-like diseases has largely focused on inhibiting the infectious ability of the self-replicating infectious protein in question or regulating the interactions of the prion or prion-like protein with regards to related conditions. An example or prior art relating to this subject is U.S. patent application Ser. No. 12/672,737, titled Treatment of Prion Protein Related Diseases, invented by John Fergus McEwan of Blakehurst, New South Wales, Australia, David Chllis-Hill of Woollahra, New South Wales, Australia, and Martin Lyndale Windsor of Randwick, New South Wales, Australia. The research which has resulted in U.S. patent application Ser. No. 12/672,737 relates to the method of administering an agent which regulates the binding of a prion or prion-like protein to associated phenomenon such as disease related Glycosaminoglycan or HSPG (Heparan sulfate proteoglycan PG Proteoglycan). Under certain conditions the Glycosaminoglycan/HSPG may be related to cancerous cells or tissue.

Various methods of chemical pharmacological stimuli are currently utilized as therapeutic cytotoxins purpose of eliminating undesired cells or tissues within an organism. The methods currently in use with regard to the current art of the sciences of oncology since the development of the first modern pharmacotherapeutic drugs designed to induce apoptosis in undesired (cancer) cells. The pharmacotherapeutic methods primarily utilized to induce apoptosis in cancerous cells work through various pathways to damage DNA, or inhibit the replication of DNA, and thus induce apoptosis upon the (attempted) replication of DNA during mitosis. While various currently utilized chemotherapeutic methods which are intended to induce cell death (apoptosis) in various forms of cancer cells are far too numerous to discuss in a befittingly rigorous manner, no currently utilized or publically proposed method of inducing cell death in cancerous cells involves the use of a self-replicating prion-like misfolded protein.

With respect to the prior art concerning the induction of cellular death in undesired (pathogenic) microorganisms, infectious fungi, and the current art of the development of antibiotic and antimicrobial drugs, no currently utilized or publically proposed method of inducing cell death in pathogenic cells involves the use of a self-replicating prion-like misfolded protein.

### BRIEF DESCRIPTION OF THE INVENTION

The invention described, illustrated, and documented herein is a new and innovative method of inducing cell death in a wide range of undesirable or infectious microorganisms, or populations consisting of the former, as well as a method of inducing cell death (or apoptosis) in certain undesirable tissues in multicellular organisms (necrosis). The method to be described within this document, is the utilization of a self-replicating, misfolded isoform of a protein specific or which is common to a particular type of cell, and which causes the death or reproductive impairment of such cell, group (colony) of cells, or tissue consisting of or partially comprising of such cells.

### DETAILED DESCRIPTION OF THE INVENTION

In accordance with 35 §112(a) a detailed description of the invention that is the subject of this application is included below. Nothing described or depicted herein should be construed as to limit the useful scope of the invention or diminish the utility of the invention with regards to an application or configuration other than the possible configuration(s) specified below.

For the purposes of this description, and any claims pursuant to such the term “prion-like” or any iteration thereof, regardless the specific phrasing, font or literary technique utilized to reference the aforementioned term (“prion-like”), shall refer to a self-replicating (proteopathic) isoform of a protein in a manner consistent with the medical or proteomic literature covering such.

An embodiment of the method which is the subject of this description may involve utilizing a self-replicating, prion like protein to specifically target a specific variety of cells, a colony thereof, or a tissue consisting of such. Certain misfolded isoforms of proteins have been observed to self-replicate (under certain conditions, both natural and artificial) in the presence of the normal (cellular) non-misfolded forms of these proteins, by the conversion of the non-misfolded form to the misfolded (prion or prion-like) isoform, resulting in an exponential increase in the misfolded form of the protein. The aggregation of the misfolded isoform of the cellular protein results in improper or impaired cellular function, resulting in the death of the (infected) cell. In the event in which a tissue contains a cell infected with such misfolded isoforms of a cellular protein, the misfolded protein will accumulate in infected cells and upon aggregation, will be transmitted to other cells, continuing the process of prion aggregation and cell death throughout the infected tissue. Prions may be transmitted from infected to non-infected tissues by multiple fluid pathways, such as blood, lymph, and cerebrospinal fluid, crossing barriers impermeable to other forms of pathogen, due to their minute size in comparison to other forms of pathogen. This method utilizes a prion-like isoform of a cellular protein found in certain undesirable cells (such as pathogenic organisms or cancer cells).

Certain (cancerous) cells produce proteins unique to that particular form of cell. Such proteins are commonly referred to in the medical literature as cancer markers or cancer marker proteins. Numerous examples of such proteins associated with individual or multiple forms of cancer cells existing within humans or animals. These proteins may be targeted as candidates for the (artificial) production of self-replicating prion-like misfolded isoforms of these cellular proteins. In the event in which these proteins are unique to their original cells, these may provide useful candidate proteins for the targeting of the cells from which they specifically originate from by the self-replicating prion-like misfolded isoforms of these cellular proteins as a practical application of the method which is the subject of this application. As many forms of cancer marking proteins exist, it rests upon the practice oncological medicine and research to determine the appropriate candidate protein to be utilized as the targeted cellular protein, as described in the method which is the subject of this application, for any particular form of cancer cell (or tissue comprising of such cells) to be destroyed by the means of this method.

This method may utilize a protein specific to a particular variety of cell (such as certain, cellular specific tumor biomarker proteins or proteins specific to certain pathogenic organisms such as Staphylococcal Protein A) this method of inducing cell death may prove to be inherently less harmful to other (normal) cells or tissues comprising of such cells, the destruction of which may be found undesirable. In the event in which this method is utilized to cause cell death in certain undesired cells found in humans or other animals (for therapeutic purposes), self-replicating prion-like isoforms of proteins specific to certain cells may be introduced either directly into the tissue(s) containing the undesired cells, or introduced into certain fluid pathways which are likely to introduce the self-replicating prion-like isoforms of the cellular protein to the tissue(s) containing the undesired cells.

Certain types of cells contain proteins unique (specific) to that variety of cell. In the event in which those cells are undesirable (such as a pathogenic microorganism or a cancer cell) these proteins may serve to enhance the utility of this method, in the elimination of these specific undesired cells or tissues comprising of the former.

Penicillin-binding proteins are a group of proteins found in the cellular wall/membranes of certain species of bacterium, including Staphylococcus strains such as Methicillin-Resistant Staphylococcus Aureus. Genetic information resulting in the production of Penicillin-binding proteins, such as Penicillin binding protein 2A (PBP2A) is encoded in the genetic material of certain organisms such as Methicillin-Resistant Staphylococcus Aureus by the mecA gene. This protein has provided Methicillin-Resistant Staphylococcus Aureus to resistance against all forms of penicillin-based antibiotics, complicating treatment procedures in the event of a Methicillin-Resistant Staphylococcus Aureus infection.

Certain embodiments of this invention may utilize the aforementioned method of utilizing a self-replicating prion-like misfolded isoform of a cellular protein (in certain embodiments this cellular protein may be a Penicillin binding protein, such as PBP2A) to convert these proteins by the process of conformational conversion into the self-replicating prion-like misfolded isoform. These self-replicating prion-like misfolded isoform may be less effective at performing the original role of the cellular protein (antibiotic resistance through binding to antibiotic chemicals) and may result in these cells affected by the self-replicating prion-like misfolded isoform (such as Methicillin-Resistant Staphylococcus Aureus) becoming more susceptible to the effects of certain chemical stimuli (such as Penicillin).

Various embodiments of the method described within this document may utilize a self-replicating prion-like isoform of a protein specific to a cellular organism to convert cellular proteins enhancing the survivability or reproductive ability of an (undesired cell) to a protein which does not enhance the survivability or reproductive ability of such a cell, through conformational shifting of the protein structures. This embodiment of the method described within this document may utilize a self-replicating prion-like isoform of a protein responsible for the resistance of certain undesired cell(s) to certain biological systems which normally function to destroy or limit the reproduction of these cells, or chemical stimuli (such as drugs) which destroy or weaken these cells, or impair the reproductive ability of such cells. The self-replicating prion-like protein may be ineffective at performing the original function of the normal cellular protein in such cells, in a manner analogous to the effect of certain prion-like proteopathic proteins found in certain fungi. This embodiment of the method which is the subject of this description will possess the benefit of enhancing the utility of certain medications or other chemicals used to damage these cells, or which impair the replication of certain undesired cells or preventing or impairing the growth of certain tissues comprising of such cells.

1) The method of inducing cell death in certain (undesired) cells exhibiting certain cellular proteins, where self-replicating prion-like isoform(s) of the cellular protein is introduced into the environment of the (undesired) cell, and induces the death of the cell through conversion of the cellular proteins to the self-replicating prion-like form.

2) The method of claim 1, where the aforementioned method is utilized to induce cell death (necrosis) in a tissue consisting of such cells.

3) The method of claim 1, where the (undesired) cells are cancerous.

4) The method of claim 1, where the cellular protein is currently known or discovered to the art of oncological research as a protein indicative of the presence of cancer cells.

5) The method of claim 1, where the aforementioned method is utilized to induce cell death (necrosis) in a tissue consisting of cancerous cells.

6) The method of claim 1, where the (undesired) cells are an infectious bacterium.

7) The method of claim 1, where the (undesired) cells are a strain of the Staphylococcus bacterium.

8) The method of claim 1, used to treat a disease.

9) The method of inducing cell death in certain (undesired) cells exhibiting certain cellular proteins, where a self-replicating prion-like isoform of the cellular protein is introduced into the environment of the (undesired) cell, and converts cellular proteins responsible for the resistance of the cell against certain chemical stimulus, into a self-replicating prion-like misfolded isoform.

10) The method of claim 9, where the undesired cell(s) are an (undesired) bacterium.

11) The method of claim 9, where the undesired cell(s) are strain of the Staphylococcus bacterium.

12) The method of claim 9, where the chemical stimulus is an antibiotic drug.

13) The method of claim 9, where the chemical stimulus is Penicillin.

14) The method of claim 9, where the undesired cells are cancerous.

15) The method of administering a dosage of self-replicating prion-like isoform(s) of a cellular protein to an organism as a means to treat, prevent, or cure a disease.
